(19) United States
(12) Patent Application Publication
(10) Pub. No.: US 2021/0100460 A1
(43) Pub. Date: Apr. 8, 2021

(54) Conformable Garment for Physiological Sensing

(71) Applicant: Massachusetts Institute of Technology, Cambridge, MA (US)

(72) Inventors: Canan Dagdeviren, Cambridge, MA (US); Irmandy Wicaksono, Cambridge, MA (US)

(21) Appl. No.: 17/032,699

(22) Filed: Sep. 25, 2020

Related U.S. Application Data
(60) Provisional application No. 62/909,673, filed on Oct. 2, 2019.

(51) Int. Cl. A61B 5/0205; G01K 13/00; G01P 15/08; G01K 7/00; G01K 1/14; G01P 15/18; G01C 19/00; G01P 1/00; A61B 5/00; A61B 5/0408; A41D 1/00; A41D 31/18

(52) U.S. Cl. CPC A61B 5/02055; A61B 5/02405; G01P 15/08; G01K 7/00; G01K 1/14; G01P 15/18; G01C 19/00; G01P 1/00; A61B 5/6804; A61B 5/0002; A61B 5/04085; A41D 1/002; A41D 31/185; A61B 2560/0214; A61B 2562/0219; A61B 2562/0271; G01K 13/002

(57) ABSTRACT

A conformable garment may fit snugly against, and may exert pressure against, skin in a region of a user's body. The garment may house multiple sensors that touch the user's skin. Each sensor may exposed to the user's skin through a hole in an inner surface of the garment. The garment may include elongated channels. Flexible, stretchable wiring may pass through a hollow central region of each channel. This wiring may provide electrical power to the sensors, and may enable wired communication between the sensors and a main hub. Each sensor may include an integrated chip and may be encapsulated in a waterproof material. Each sensor may output electrical signals that encode digital data and that are transmitted, via the wiring, to a main hub housed in the garment. The encapsulated sensors and the wiring may remain in the garment when the garment is washed.

### RELATED APPLICATIONS

This application claims the benefit of U.S. Provisional Application No. 62/909,673 filed Oct. 2, 2019 (the “Provisional”).

### FIELD OF TECHNOLOGY

The present invention relates generally to conformable garments that house physiological sensors.

### COMPUTER PROGRAM LISTING

The following three computer program files are incorporated by reference herein: (1) data_stream.txt with a size of about 13 KB; (2) plot_graph.txt with a size of about 5 KB; and (3) plot_temp2d.txt with a size of about 15 KB. Each of these three files were created as an ASCII .txt file on Aug. 26, 2020.

### SUMMARY

In illustrative implementations of this invention, a conformable garment houses sensors that measure physiological parameters of a human user.

The garment may include one or more elastic, stretchable materials which cause the garment—and sensors housed in the garment—to fit snugly against the skin of the user who is wearing the garment. Furthermore, the dimensions of the garment may be customized (e.g., tailored) to help achieve a snug fit. Put differently, for each particular user: (a) measurements may be taken of the size of different parts of the particular user's body; and (b) a conformable garment may be fabricated with dimensions that are selected, based on the measurements, to help cause the garment to fit closely to the particular user's body. For instance, in some cases, at least some of the dimensions of the garment are —when the garment is not being stretched—are up to 30% smaller than the corresponding measured dimensions of the user's body.

The conformable garment may be any type of clothing or wearable item that compresses and fits snugly against a user's skin. For instance, the conformable garment may comprise a snug-fitting item of clothing (or a snug-fitting wearable) that is a bodysuit, suit, shirt, top, tank, pants, belt, sock, glove, undershirt, underpants, wrist-band, ankle-band, arm-band, leg-band, head-band, hat or cap.

Each sensor may be housed in the garment in such a way that the sensor is directly exposed to, touches, and fits snugly against skin of the user who is wearing the garment. In some cases, each sensor is located on an inner surface of the garment. In some cases, each sensor touches the user' skin because the sensor protrudes through, or is exposed on or through an inner surface of the garment. For instance, an inner surface of the garment may include openings which expose respective sensors housed in the garment.

To help cause the sensors to touch and to fit snugly against the skin of the user, the garment may exert a compressive force against skin of the user. For instance, the compressive force may be exerted by one or more elastic, stretchable materials of the garment. In some cases, the compressive force exerted by the garment against a region of skin of the user is more than 1 mmHg and less than 41 mmHg. A compressive pressure of 2 mmHg may be sufficient to ensure achieve a snug fit of a sensor against skin, whereas pressures lower than that may not. A compressive pressure greater than 44.1 mmHg may unduly restrict blood circulation, whereas lower pressures may not. (In some persons, average capillary blood pressure is about 32 mmHg near the skin—e.g., in the dermis). A compressive pressure that is greater than or equal to 10 mmHg and less than or equal to 40 mmHg may be desirable, in order to achieve a fit that is comfortable for the user, and to ensure that sensors remain in snug contact with the user's skin under a wide range of circumstances. A compressive pressure that is greater than or equal to 20 mmHg and less than or equal to 30 mmHg may be desirable, in order to achieve a fit that is even more comfortable for the user, and to ensure that sensors remain in snug contact with the user's skin under even a wider range of circumstances.

We sometimes describe the garment as “conformable”, because, when worn by a user, the garment may snugly fit, and press against, a user's skin in such a way that an inner surface of the garment conforms to the shape of a portion of the user's body.

In some cases, the sensors are not visible on or through the outer surface of the garment. Thus, in some cases, all or almost all of the outer surface of the garment looks like that of a “normal” sensor-less garment

In illustrative implementations of this invention, multiple sensors are housed in or on a garment. For instance, in some cases, the number of sensors housed in or on a garment is greater than or equal to two and less than or equal to one thousand.

In illustrative implementations of this invention, one or more types of sensors are housed in or on a garment. For instance, a garment may house one or more of the following types of sensors: temperature sensor; accelerometer; gyroscope; inertial measurement unit; electrodermal activity (EDA) sensor; photoplethysmography sensor; pulse oximeter; electrocardiography (ECG) sensor; surface electromyography sensor, blood pressure sensor, perspiration sensor, and electroencephalography (EEG) sensor.

As a non-limiting example, a compressive garment may, in some use scenarios, house: (a) multiple (e.g., more than one and less than one thousand) temperature sensors; and (b) one or more accelerometers, gyroscopes and/or IMUS. The multiple temperature sensors may together perform distributed temperate sensing at many locations on the exterior of the user's body. The accelerometer(s), gyroscope(s) and/or IMU(s) may employed for ballistocardiography or seismocardiography readings. For instance, measurements taken by the accelerometer(s), gyroscope(s) and/or IMU(s) while the user wears the garment may be analyzed to measure user's heart rate, heart rate variability, or heart waveform. Furthermore, the accelerometer(s), gyroscope(s) and/or IMU(s) may be employed for detecting respiration rate, respiration waveform, posture, motion, or activity level.

In some cases, multiple sensor modules are housed at different positions in or on the garment. Each sensor module may include one or more of the sensors.

Each sensor module may be connected by one or more wired electrical connections to a main hub that is housed in the garment or that is otherwise worn by the user. These wired connections may enable two-way, wired communication between each sensor module and the main hub, and may deliver electrical power from a power source. The power source may be located in the main hub or located elsewhere (e.g., worn elsewhere on the user's body). The wired connections may comprise ribbons or cables. Each ribbon or cable may include multiple (e.g., more than one and less than one hundred) flexible conductive tracks or wires. These conductive tracks or wires may comprise copper, silver, metal or other conductive material. In each ribbon or cable, the conductive tracks or wires may be coated with, separated by, and/or located between, one or more insulative materials. The insulative material(s) (e.g., polyimide) may be more rigid than the conductive tracks or wires (in order to provide support), but may still be flexible and stretchable.

The wired connections may comprise hardware of one or more communication buses, such as any serial communication bus, parallel communication bus, synchronous communication bus, packet-switched communication bus, or single-ended communication bus. The sensors and a computer (e.g., microprocessor) in the main hub may be programmed to communicate in accordance with a protocol that corresponds to the communication bus hardware. For instance: (a) the wired connections may comprise hardware for an I2C (inter-integrated circuit) communication bus; and (b) the sensors and microprocessor in the main hub may be programmed to communicate in accordance with an I2C protocol.

We sometimes call a wired connection between a sensor module and the main hub an “interconnect”. Each interconnect may be elongated.

Each interconnect may be configured to elastically bend and stretch. For instance, each interconnect may be configured to elastically stretch (undergo elastic deformation), when undergoing tensile stress, in such a way as to increase length of the interconnect. For instance, when undergoing tensile stress, each interconnect may elastically stretch in such a way as to increase end-to-end length of the interconnect (i.e., straight Euclidean distance from a first end to a second end of the interconnect) by up to 25%, up to 30%, up to 35%, up to 40%, up to 45%, up to 50%, or up to 60%, relative to end-to-end length of the interconnect when the interconnect is not undergoing any tensile stress.

Each interconnect may have a geometric shape that facilitates elastic flexing and stretching. For instance, in some cases, each interconnect has a serpentine geometric shape when not under tensile stress. Tensile stress may tend to cause an interconnect to straighten. For instance, as tensile stress on a serpentine interconnect increases, the curvature of the bends of the interconnect may decrease, causing the overall shape of the interconnect to become more similar to a straight line. Later, when tensile stress on the serpentine interconnect is released, the curvature of the bends of the interconnect may increase, causing the interconnect to return to its initial serpentine shape.

Each sensor may include an integrated chip and may output electrical signals that encode digital data. For instance, at least some of the digital data may represent measurements taken by the sensor or may represent information derived from these measurements. Alternatively, all or at least some of the sensors may output analog signals. For example, at least some of the analog signals may represent measurements taken by the sensor or may represent information derived from these measurements.

Each sensor module may be waterproof, because it includes one or more layers of one or more waterproof materials. For instance, these waterproof layers may be include: (a) one or more layers of DuPont® PE773, and (b) one or more layers of thermoplastic polyurethane (TPU). These waterproof layer(s) may prevent water from entering an interior region of the sensor where electronic components (e.g., an integrated chip) are located. The waterproof layers may have one or more openings for the interconnects. The waterproof layers may fit tightly around each interconnect, creating a seal around the interconnect in the region where the interconnect passes through the waterproof layer(s). In some cases, the outermost waterproof layer of each sensor touches the user when the user is wearing the garment.

As noted above, the main hub may be housed in the garment or otherwise worn by the user. The main hub may comprise a computer (e.g., a microprocessor), a wireless module, a power source and housing. The computer may control when the respective sensors take measurements (e.g., by outputting signals that cause the respective sensors to turn on and off). Furthermore, the computer may calculate physiological parameters of the user in real time, by filtering, processing and/or analyzing data that represents measurements taken by the sensors. The computer may output signals that encode these physiological parameters. These signals may be transmitted by a transceiver in the wireless module. For instance, the wireless module may transmit and receive wireless radio signals in accordance with a Bluetooth® communication protocol. The power source may comprise one or more batteries (e.g., one or more lithium-polymer batteries).

In illustrative implementations, the garment is washable. The sensor modules may be encapsulated and waterproof, and the electrical interconnects may be waterproof. Thus, the sensor modules and interconnects may remain in the garment when the garment is washed.

In some cases, all (or at least a portion) of the main hub is removed from the garment during washing and then reinserted into the garment after it has dried. The main hub may be housed in a pocket or pouch of the garment. The pocket or pouch may be opened in order to insert or remove the main hub, and may be closed when the main hub is inside the pocket or pouch. For instance, the pocket or pouch may be closed with a snap, zipper or hook-and-loop fastener (such as a Velcro® fastener). The main hub may be configured to easily detach from, and then re-attach to, electrical leads for the interconnects.

The garment may include hollow channels through which the interconnects are threaded. Each channel of the garment may be elongated, and may have an elongated central cavity. Put differently, each channel of the garment may comprise a tube (e.g., a knitted tube). In some cases, each channel has many holes in its sides (e.g., small holes at interstices between knitted yarn or between woven threads). In some cases, each channel of the garment comprises a knit fabric, woven fabric, non-woven, non-knit fabric, or other material.

Each interconnect may, over most or all of its length, be located inside a channel of the garment. In some cases, one or more interconnects are located in each channel.

The garment may also include strips of material that are positioned between the hollow channels. For instance, a strip of material may be located between, and thus may physically separate, each pair of adjacent channels. One or more of the strips may be elongated. Each of these strips of material may be connected (e.g., by knitting or weaving) to the channel(s) that it adjoins. Thus, the garment may be an integral object that includes both the strips of material and the channels.

In many cases, the sensor modules: (a) are each rigid or semi-rigid; and (b) each include rigid or semi-rigid electronic components (e.g., components that include metal or semi-conductor parts). Likewise, in many cases, the main hub: (a) is rigid or semi-rigid; and (b) includes rigid or semi-rigid electronic components (e.g., components that include metal or semi-conductor parts).

Alternatively, in some cases, one or more of the sensors comprise a textile or fabric. For instance, one or more of the sensors may include a conductive yarn or conductive thread that is employed for resistive or capacitive sensing. Likewise, one or more of the interconnects may comprise, in whole or part, one or more conductive yarns or conductive threads.

The garment may comprise a knit fabric, woven fabric, non-woven, non-knit fabric, or any other material, or any combination of the above. For instance, in some cases, at least the channels are knitted. In some cases, both the channels and the strips between them are knitted. If a knit fabric is employed, the knit structure of the fabric may cause the garment to be elastic and to exert a compressive force against the user.

The garment may comprise artificial or natural fibers, yarns or threads, or any combination of the above. For instance, the material of the garment may comprise a high-flex, polyester fabric.

In some implementations of this invention, a washable, comfortable, personalized-fit electronic textile garment (e.g., bodysuit) measures physiological parameters. The sensors may be distributed in many locations on a user's body. Advantageously, this single garment may be employed instead of multiple wearables, since its sensors can cover a large region of the body to perform spatiotemporal physiological and physical activity sensing. Sensor-embedded fabrics may be cut in any size, joined, and tailored to create a garment for various needs and applications. In some cases, the sensor-embedded fabrics are fabricated by roll-to-roll manufacturing.

The Summary and Abstract sections and the title of this document: (a) do not limit this invention; (b) are intended only to give a general introduction to some illustrative implementations of this invention; (c) do not describe all of the details of this invention; and (d) merely describe non-limiting examples of this invention. This invention may be implemented in many other ways. Likewise, the Field of Technology section is not limiting; instead it identifies, in a general, non-exclusive manner, a field of technology to which some implementations of this invention generally relate.

### BRIEF DESCRIPTION OF THE DRAWINGS

FIG. 1 shows a conformable garment with integrated sensor modules.

FIG. 2A shows a sensor module, interconnects and a channel.

FIGS. 2B and 2C show a channel.

FIG. 3 shows an exploded view of a sensor module.

FIG. 4 shows a region of a conformable garment.

FIG. 5 shows a sensor module with serpentine interconnects.

FIG. 6 shows a serpentine interconnect.

FIGS. 7, 8 and 9 show part of a flexible serpentine interconnect stretched by different amounts.

FIG. 10 is block diagram of hardware employed for modular sensing with a conformable garment.

FIG. 11 is a cross-sectional view of a conformable garment with integrated electronics.

FIG. 12 is a flowchart for a method of processing sensor measurements.

The above Figures are not necessarily drawn to scale. The above Figures show illustrative implementations of this invention, or provide information that relates to those implementations. The examples shown in the above Figures do not limit this invention. This invention may be implemented in many other ways.

### DETAILED DESCRIPTION

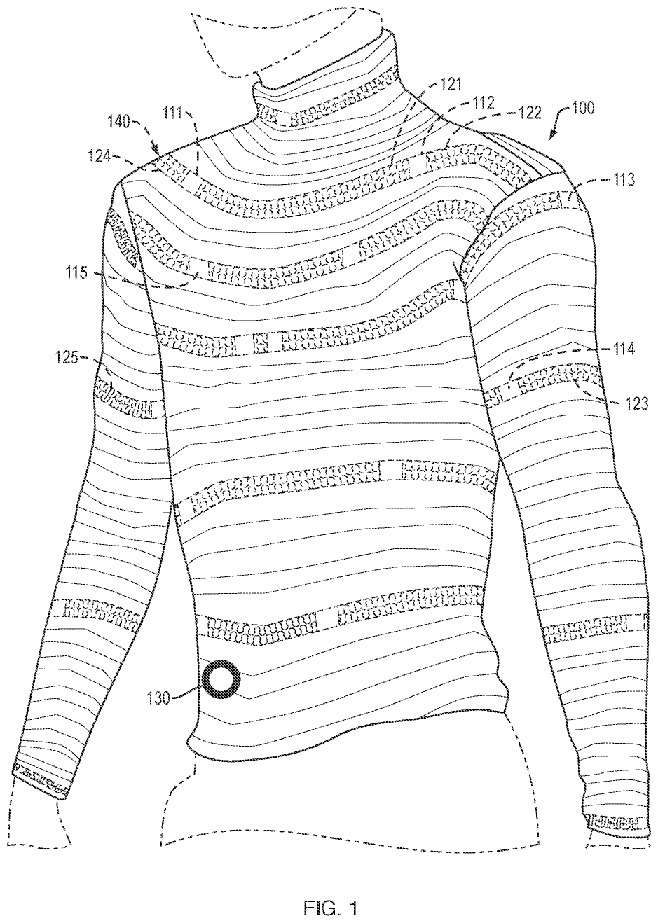

FIG. 1 shows a conformable garment with integrated sensor modules, in an illustrative implementation of this invention. In FIG. 1, conformable garment 100 comprises a bodysuit that covers regions of a user' body, including the torso, part of the arms and part of the neck. Garment 100 fits snugly against, and exerts a compressive force against, the user's skin in these regions of the user's body. Garment 100 houses sensor modules (e.g., 111, 112, 113, 114, 115) and wired interconnections (interconnects) (e.g., 121, 122, 123, 124, 125). Garment 100 has an inner surface that touches the user's skin and an outer surface that is on the other side of the garment. Put differently, the outer surface of the garment is farther from the user's skin than is the inner surface of the garment.

The sensor modules and interconnects are shown in FIG. 1, for ease of illustration. However, they may actually be hidden from external view, because they are covered by the garment's outer surface. At the inner surface of the garment, however, at least a portion of each sensor in each sensor module is exposed to, touches, and fits snugly against the user's skin.

In FIG. 1, garment 100 includes elongated channels. These channels may be knitted. A set of interconnects may be housed in the channels. Each interconnect in the set may be inside—and extend approximately parallel to—a longitudinal axis of a channel. The interconnects may enable the sensor modules to communicate with a main hub 130 and provide power to the sensor modules. Each wired interconnect may include multiple wires and a flexible insulative sheath. The sheath may cover the wires and separate them from each other.

In FIG. 1, the channels are arranged in approximately horizontal elongated strips. Each such strip may include one or more sensor modules and wired interconnects. For instance, horizontal strip 140 includes two sensor modules 111, 112 and three wired interconnects 121, 122, 124. Alternatively, the strips (including sensor modules, interconnects, and channels) may be oriented in any direction (e.g., vertical, slanted or horizontal) and may be straight or curved.

In FIG. 1, a main hub 130 includes a microprocessor, power source, and wireless module.

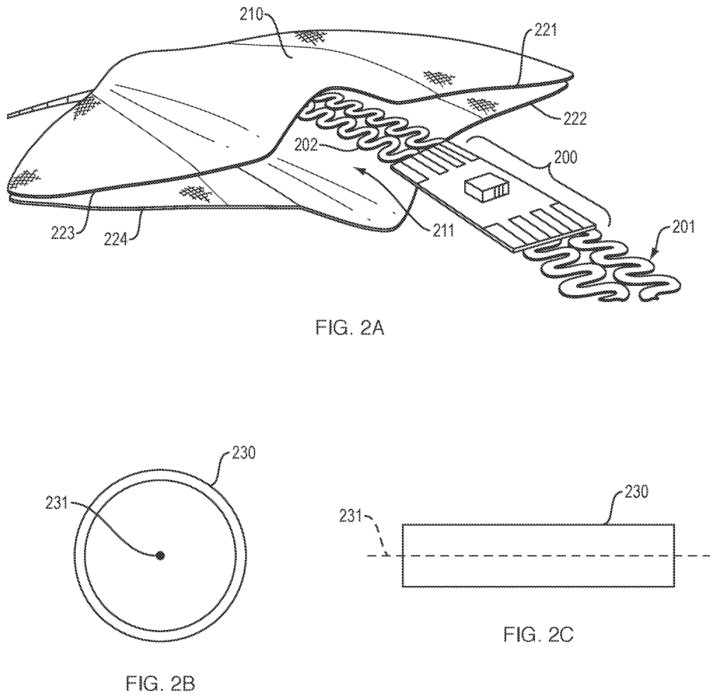

FIG. 2A shows a sensor module, interconnects and a channel, in an illustrative implementation of this invention. In FIG. 2A, a sensor module 200 is electrically connected to serpentine interconnects 201, 202. An elongated channel 210 of the garment has an elongated hollow region 211 in the center of the channel. This elongated hollow region is aligned with, and runs along, a longitudinal axis of the channel. Interconnect 202 is positioned inside the hollow region 211 of channel 210 and is aligned with, and runs along, a longitudinal axis of channel 210. Channel 210 is located between a first region formed by fabric 221 and fabric 222 and a second region formed by fabric 223 and fabric 224. Fabrics 221, 222 may be interlocked to form a single integral layer. Likewise, fabrics 223, 224 may be interlocked to form a single integral layer. Channel 210 and fabrics 221, 222, 223, 224 (in FIG. 2A) and channel 230 (in FIGS. 2B and 2C) may each be knitted.

As noted above, in some cases, the sensors include one or more accelerometers, gyroscopes, IMUs, and temperature sensors. For instance, each accelerometer and gyroscope may comprise a digital 3 axis accelerometer and digital 3-axis gyroscope, respectively. In some cases, each accelerometer comprises a proof mass (also known as damped mass) on a spring. In some cases, in each accelerometer, an electronic or electrical circuit detects motion of the proof mass (e.g., by electrical, piezoelectric, piezoresistive, or capacitive measurements). In some cases, in each accelerometer, a motor (e.g., electromagnetic or electrostatic) prevents the proof mass from moving too far. In some cases, each accelerometer comprises a MEMS (micro-electro-mechanical system). For instance, each MEMS accelerometer may comprise a proof mass at the end of a cantilever beam, and motion of the proof mass may be damped by residual gas in the MEMS. In some cases, each gyroscope comprises a MEMS gyroscope, a vibrating structure gyroscope (VSG), a fiber optic gyroscope, a hemispherical resonator gyroscope (HRG), a dynamically tuned gyroscope (DTG), or a ring laser gyroscope. In some cases, each IMU comprises one or more accelerometers, gyroscopes and magnetometers. In some cases, each temperature sensor is an electronic or electrical sensor and includes one or more thermistors, thermocouples, resistance thermometers or silicon bandgap temperature sensors.

FIGS. 2B and 2C show a longitudinal axis 231 of a channel 230.

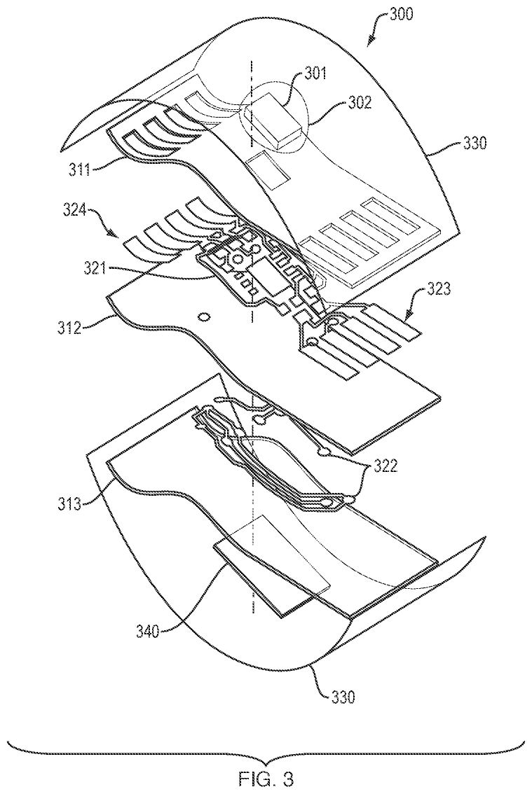

FIG. 3 shows an exploded view of a sensor module, in an illustrative implementation of this invention. In FIG. 3, the sensor module 300 is covered by an outer sheath of TPU 330 and includes a stiffener 340. In some cases, stiffener 340 comprises polyimide or aluminum sheet. Inside the sensor module, a sensor integrated circuit 301 is encapsulated by a waterproof encapsulant 302. For instance, the encapsulant may comprise DuPont® PE773. Also, inside the sensor module, two layers 321, 322 of copper are sandwiched between three layers 311, 312, 313 of a relatively stiff insulator (e.g. polyimide). Copper layer 321 includes a first set of four copper pads 323 and a second set of four copper pads 324. The first set of four pads 323 may be joined with solder to four wires in a first interconnect; and the second set of four pads 324 may be joined with solder to four other wires in a second interconnect. Alternatively, the copper may be replaced by any other conductive material, such as silver.

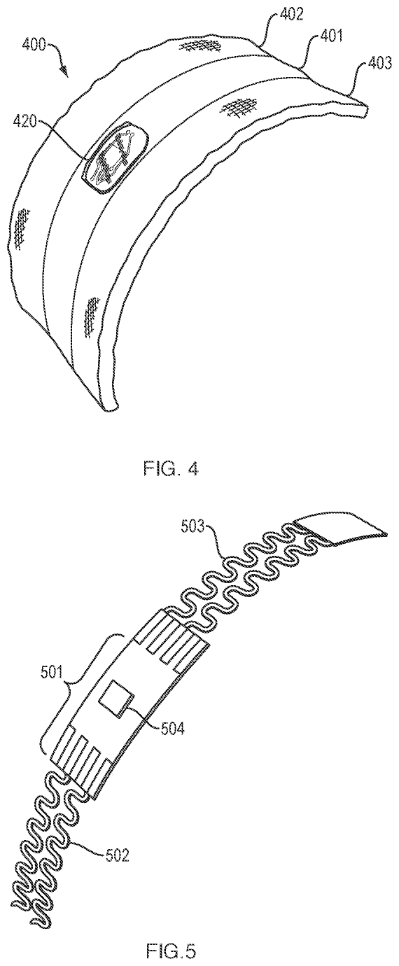

FIG. 4 shows a region 400 of a conformable garment, in an illustrative implementation of this invention. This region 400 includes a first elongated strip of fabric 401 and two other elongated strips 402, 403 of fabric. The first strip 401 is positioned between, and is touching, the other two strips 402, 403. For instance, the first strip may be attached to the other two strips by knitting, weaving, chemical bonds or other mechanical attachment. The first strip 401 includes one or more channels. Each channel has, in its center, an elongated hollow region. This elongated hollow region is aligned with, and runs along, a longitudinal axis of the channel. The other two elongated strips 402, 403: (a) may each comprise a single layer of interlocked, knitted material; (b) may each, if laid on a flat surface, be substantially flat; and (c) each do not have an elongated, internal hollow that is configured to house a wired interconnect. All of the fabric in region 400 may be knitted, such as by a digital knitting machine. In FIG. 4, a sensor module 420 is housed in the first elongated strip 401. This sensor module 420 is shown in FIG. 4 for ease of illustration, but may actually be hidden from external view, because it is beneath the outer surface of the garment. At least a portion of sensor module 420 is exposed at, or protrudes through, or is on, the internal surface (not shown in FIG. 4) of the garment.

FIG. 5 shows a sensor module 501 with serpentine interconnects 502, 503, in an illustrative implementation of this invention. Sensor module 501 includes an integrated circuit 504.

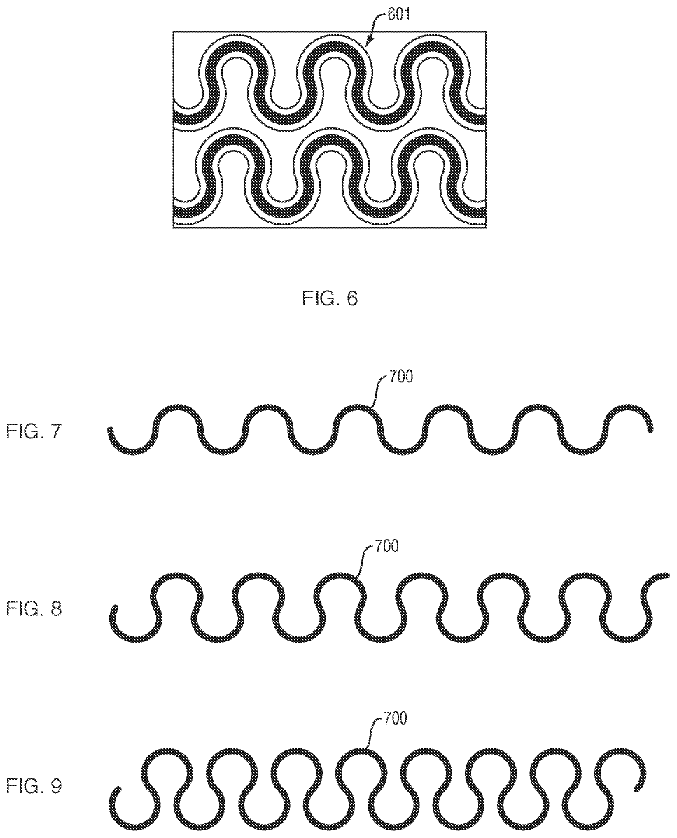

FIG. 6 shows a serpentine interconnect 601, in an illustrative implementation of this invention. In FIG. 6, serpentine interconnect 601 comprises (a) multiple conductive wires and (b) an insulative sheath that is sufficiently stiff to provide some support but is still somewhat flexible. For instance, the insulative sheath may comprise polyimide.

FIGS. 7, 8 and 9 show a ribbon 700 of a serpentine interconnect stretched by different amounts, in an illustrative implementation of this invention. For instance, ribbon 700 may comprise conductive wires that are sheathed in, and separated by an insulator such a polyimide. Ribbon 700 is stretched more in FIG. 7 than in FIG. 8 (due to greater tensile stress on the ribbon in FIG. 7 than in FIG. 8.) Likewise, ribbon 700 is stretched more in FIG. 8 than in FIG. 9 (due to greater tensile stress on the ribbon in FIG. 8 than in FIG. 9.)

In illustrative implementations, the stretchable interconnections may be strained without undergoing fracture. For instance, in some cases, the interconnections may undergo strain of greater than 0.4% (e.g., greater than 0.5% and less than or equal to 1%, greater than 1% and less than or equal to 5%, greater than 5% and less than or equal to 10%, greater than 10% and less than or equal to 15%, greater than 15% and less than or equal to 25%, greater than 25% and less than or equal to 30%, greater than 30% and less than or equal to 40%, greater than 40% and less than or equal to 50%, greater than 50% and less than or equal to 60%, or larger) without fracturing. In some cases, stable island structures (e.g., device components such as sensor modules) are configured to deform (e.g., by compression, elongation and/or twisting) without fracturing.

A substrate of the interconnects (or of the sensor modules or of both the interconnects and sensor modules) may be a material with a Young's modulus that is less than or equal to 55 MPa, such as less than or equal to 50 MPa, less than or equal to 30 MPa, or less than or equal to 25 MPa. The thickness of the substrate may vary depending upon, among other things, the application and the properties of the material. For instance, in some cases, the substrate has a thickness of: (a) greater than 10 nm and less than 10 μm; or (b) greater than 50 nm and less than 5 μm; or (c) greater than 100 nm and less than 1 μm. In some cases, the substrate comprises a plastic or polymer.

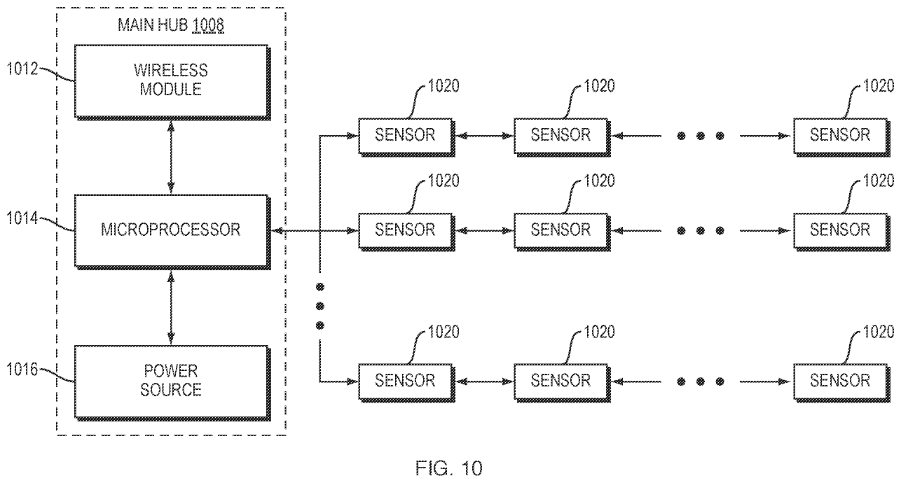

FIG. 10 is a block diagram that illustrates hardware employed for modular sensing with a conformable garment, in an illustrative implementation of this invention. In FIG. 10, a main hub 1008 includes a microprocessor 1014, power source 1016 and wireless module 1012. The main hub may be housed in or attached to the conformable garment. The conformable garment may house multiple sensors (e.g., sensors 1020). Power source 1016 may comprise one or more batteries.

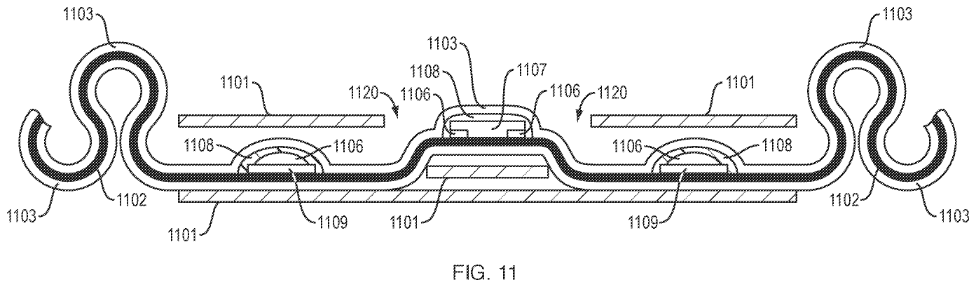

FIG. 11 is a cross-sectional view of a conformable garment with integrated electronics, in an illustrative implementation of this invention. In FIG. 11, knitted walls 1101 form a fabric channel. Interconnects are threaded through a hollow region in the center of this channel. An opening 1120 is located on the inner surface of the garment, in the walls of the channel. This opening 1120 exposes a sensor module. The sensor module includes an integrated circuit (IC) 1107. The IC 1107 is encapsulated by a waterproof polymer encapsulant 1108, such as DuPont® PE-773, and is attached to a polyimide layer(s) 1102 by a low-temperature solder 1106. Wires are embedded in the polyimide layer(s). These wires extend outward from the sensor module and attach to, or flatten into, pads 1109. In turn, the pads 1109 are attached by solder 1106 to wires of interconnects. Outer layer(s) of TPU 1103 may surround and protect the encapsulant 1108 and the polyimide 1102.

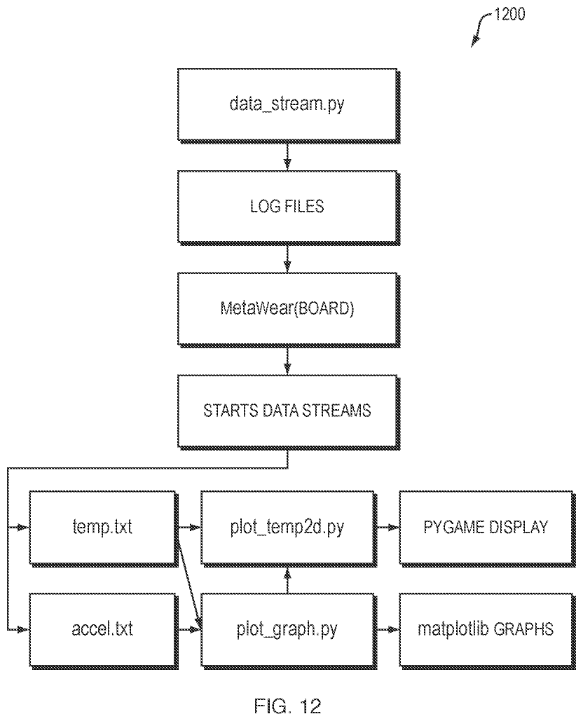

FIG. 12 is a flowchart for a method 1200 of processing sensor measurements.

Prototype

The following 28 paragraphs describe a prototype of this invention.

In this prototype, a suit with integrated sensors monitors human skin surface temperature distribution, heart-rate, and respiration. The suit is tailored from a fabric that is integrated with an assortment of sensor integrated circuits (ICs) and interconnects in the form of flexible-stretchable electronic strips. The textile platform comprises channels or pockets for the weaving of these electronic strips. The sensor ICs and interconnects are developed using two-layer industrial flexible printed circuit board (PCB) processes with additional steps for chip and passive component assembly and encapsulation with thermoplastic polyurethane (TPU) and washable encapsulant.

In this prototype, a tailored, electronic textile conformable suit performs large-scale, multi-modal physiological (temperature, heart rate, and respiration) sensing in vivo. This platform may be customized for various forms, sizes and functions using standard, accessible and high-throughput textile manufacturing and garment patterning techniques. Similar to a compression shirt, the soft and stretchable nature of the tailored suit causes intimate contact between electronics and the skin with a pressure value of around 25 mmHg, allowing for physical comfort and improved precision of sensor readings on skin. Sensors housed in the suit may detect skin temperature with an accuracy of 0.1° C. and a precision of 0.01° C., as well as heart-rate and respiration with a precision of 0.0012 m/s2 through mechano-acoustic inertial sensing. The knit textile electronics may be stretched up to 30% under 1000 cycles of stretching without significant degradation in mechanical and electrical performance. The sensor system housed in the suit may simultaneously and wirelessly monitor 30 skin temperature nodes across the human body over an area of 1500 cm2, during seismocardiac events and respiration, as well as physical activity through inertial dynamics.

In this prototype, the sensor modules are electrically connected to the interconnects by conductive pads that are soldered together. The interconnect strips have multiple islands of pads with an area of 1 mm×4 mm in between serpentine interconnects. The pad design enables the interconnect strips to be reconfigurable. Each interconnect strip may be cut and joined to any length needed for connection to the sensor modules. Female headers or holes at the end of these interconnect strips may be used for textile-hardware connections by looping conductive threads or thin wires.

In this prototype, a main module provides electrical power to the sensor modules, processes data from the sensors in the sensor modules, and performs wireless communication.

In this prototype, all of the sensor modules are electrically connected to a main module through an I2C (inter-integrated circuit) bus interface with four signal wires (VDD, SCL, SDA, GND). The I2C bus accesses 128 addresses. The prototype may, in some use scenarios, handle up to 32 temperature sensors (0x40 to 0x5f in 7-bit address) and 2 inertial measurement units (IMU) (0x68 and 0x69) with minimal wirings. Because every sensor module takes its own measurements, performs processing locally, and has a unique sensor address, adding several sensor nodes of different nature does not introduce cross-talk.

In this prototype, a modular sensor network is embedded in a fabric. The sensor modules are positioned in horizontal rows, in such a way that each horizontal row includes multiple sensor modules. In each horizontal row, the sensor modules in the row are mechanically and electrically connected to each other by the interconnects. Data representing measurements by the sensors in the sensor modules are sent via wires in the interconnects to a main hub. The main hub comprises a Bluetooth® low-energy (BLE) module, a microprocessor, and a power source. In this prototype, as more fabrics and sensors are joined to the main hub, I2C address scanning from a microcontroller shows an increase of number of sensor addresses detected.

In this prototype, seismocardiography (SCG) records subtle motions around the body due to the atrial muscle contractions and blood ejection as the heart pumps. The frequency characteristic waveform of SCG thus reflects cardiac mechanical events. In this prototype, SCG is unobtrusively monitored by attaching IMUs to the body or integrating them to objects that physically touch the body. In some use scenarios, the IMUs are located on the user's body in such a way that they also capture body motions caused by the contraction and dilation of the lungs, which relate to the breathing mechanism. In a use scenario of this prototype, an IMU is placed on the user's skin below the sternum. This position, immediately below the sternum, is a sensitive location for detecting both heart and breathing activities. Also, an accelerometer sensor module is encapsulated and integrated into a fabric patch.

In this prototype, SCG data are given by the accelerometer z-axis value, with a sensitivity setting of 2 g, precision of 0.0012 m/s2, and a sampling frequency of 100 Hz. A finite impulse response (FIR) low-pass filter may eliminate low-frequency signals caused by respiratory waveforms. A computer may analyze the SCG data to detect, among other things, mitral valve closure, aortic valve opening, rapid ventricular ejection occurring right after R-peak or ventricle depolarization and aortic valve closure, mitral valve opening (MO), rapid ventricular filling after T-peak, and ventricle relaxations.

In this prototype, in order to detect the respiratory waveform, a FIR (finite impulse response) low-pass filter eliminates high-frequency signals due to heart-beat events and obtains a direct current (DC) component of the signals.

In this prototype, digital knitting is employed to fabricate the fabric. The digital knitting is a programmable, automatic machine process of stitching interlocked loops from multiple strands of yarn. In the digital knitting, several needles or hooks arrange the interlocking mechanism of loops into fabrics. The digital knitting starts with multiple cones of yarn that gets pulled into the machine by yarn carriers until a certain pre-programmed tension is achieved. The carriers then slide back-and-forth horizontally while the needles catch the yarns to form the loops. Each carrier may be sequentially controlled to slide and combine different yarns to form structural or color patterns. The programming interface for the digital knitting includes two grid sections. The left grid is used to develop the shape and pattern of the knit fabrics through x-y color block programming, where each color and logo represent specific knit operation.

In this prototype, a flat two-bed digital knitting machine patterned textile channels (in which the interconnects are later placed) by employing a combination of two-layer jersey and interlocked knitting. As a non-limiting example, the digital knitting produced a region of fabric that includes four textile channels and three interlocked stripes. In this region, single-color layers each comprise two layers of separated fabric, while dotted stripes are each formed by interlocked patterns, which combine two fabric layers into one. As another non-limiting example, the digital knitting produced three fabrics with the size of 55 by 120 cm: one for front-side, one for back-side, and one for a pair of long-sleeves. In these three fabrics, channels with an internal width of 1 cm provide enough room for interconnects and sensor modules, which have a width of 0.6 cm. In these three fabrics, the distance between each sensor is at least 1 cm vertically based on the channel width, and at least 2 cm horizontally based on the interconnect module length.

In this prototype, after the whole fabric is digitally knitted, it is cut for different body parts of a user based on measurements of dimensions of the user. Electronic-textile integration is then performed, by threading the electronic strips into the textile channel. Then the sensor-integrated fabrics are then sewn into a bodysuit to form a garment.

In this prototype, data from sensors in the sensor modules in each of the respective horizontal interconnect strips is sent, according to the I2C protocol, to the main hub via four thin vertical copper wires that pass through seams in the garment. The main hub includes a microprocessor, BLE (Bluetooth@ low energy) module, and rechargeable lithium polymer battery in a compact form. The lithium polymer battery is rated at 3.7 V, 100 mAh and has a two-hour charging time. The total current consumption while the main module and all of the sensor nodes are active is approximately 18.6 mA. The battery is rated at 100 mAh, sufficient to power electronics (e.g., main hub and sensor modules) in the garment for approximately 5 hours and 20 minutes. Conductive snaps function as a textile-hardware connector to link the I2C pins on the microprocessor with the I2C wires in the textile. The pluggable mechanism allows the wireless communication and main processing hardware to be removed during charging of the battery. The I2C pins of this micro-controller are wired to the conductive snaps for the textile-hardware interface. Wireless BLE communication transmits, to a wireless transceiver that is remote from the garment, data representing or derived from measurements taken by the sensors housed in the sensor modules in the garment. The remote wireless transceiver, in turn, sends the data to a remote computer that accesses all of the sensor addresses and logs their data accordingly. This data may then be stored or visualized in real-time using software that includes or calls upon Python® Matplotlib and Pygame library.

In this prototype, the dimensions of the garment may be personalized to ensure there is sufficient pressure for sensor contact between the textile and skin of a particular user. In this prototype, the garment exerts a compressive pressure against at least a region of a user's body. In some cases, this pressure is greater than or equal to 2 mmHg and less than or equal to 44.1 mmHg. Pressure in this range may ensure a reliable contact between the sensors and the skin.

In this prototype, a pressure of 2 mmHg may be sufficient to accurately measure skin temperature in some use scenarios, while a larger pressure of up to 20 mmHg may result in an increase of temperature due to the pressure exerted to the local tissue. In this prototype, for wearable comfort, the compression pressure is preferably not more than 44.1 mmHg, which is close to the average capillary blood pressure of 32.3 mmHg near the skin.

In this prototype, measurements are taken of a user's body, in order to customize dimensions of the garment to snugly fit the user's body.

In a test of this prototype, the extension (stretch/strain) of three stretchable interconnects did not influence their resistances (0.32Ω to 0.45Ω) until rupture events at strain values around 79 to 88%.

This prototype is washable for long-term use.

In this prototype, the 100% high-flex polyester fabric is breathable, allowing moisture vapor to pass through the fabric.

In this prototype, distributed temperature sensors may be employed to monitor temperature change around the body during various dynamic physical activities such as daily activity and exercise, to see how heat dissipation and perspiration influence thermal comfort or athletic performance.

In this prototype, a temperature sensor module includes a two-layer flexible PCB (printed circuit board) with 18 μm thick Cu traces, 28 μm thick base polyimide (PI) substrate, and 28 μm thick PI outer shell. The temperature sensor IC (integrated circuit) is 850 μm in thickness. To fabricate the temperature sensor module, the temperature sensor IC is soldered into the pads with 75 μm thick PI stiffener as a support structure and encapsulated with 150 μm thick washable encapsulant. The entire module is then encapsulated in a TPU shell with 100 μm thickness for each top and bottom layer.

In this prototype, fabric of the garment is knitted. A digital flat two-bed knitting machine produces the knitted material. Two yarn carriers are used in order to make two layers of weft-knit fabric. In this weft-knit fabric, the loops are made in a horizontal way from a single yarn. The digital two-bed knitting machine produces a single layer region of fabric by interlocking. To achieve this interlocking, two sets of needles in the knitting machine knit back-to-back in an alternate sequence to create two sides of the fabric that are exactly in line with each other, forming one layer. Each yarn carrier holds 2-ply (75 denier each ply) of high-flex polyester yarns. Textile channels for electronic integration are knitted by allowing both the front and back needle beds to knit simultaneously and by making a spacer fabric with a hollow channel. The number of wale lines, which is 20 in this spacer fabric, defines the width of the opening of around 1 cm while the course line number defines the width of the entire knit fabric. The rest of the fabric is formed through interlocking. Solder-tip melting is performed to open the channels for the exposed part of the sensor modules with a distance of 1.5 cm.

In this prototype, the knitted fabric is laser-cut with the open channels positioned in a horizontal orientation. The horizontal measurements (e.g. neck circumference, waist circumference, thigh circumference) are reduced by around 10% depending on the dimension to ensure a tight fit. A seam allowance of 1.5 cm is used on the pattern pieces. The garment comprises a front, a back, two sleeves, and polo neck pieces. The raw edges of the seams are joined together using a zig-zag stitch with a sewing machine as an overlocking stitch.

In this prototype, after the sensor-interconnects modules bonding by hot-melt soldering, the sensor electronics are encapsulated using medical and semiconductor grade epoxy resin that is machine-washable for both mechanical and electrical protection. The electronic strips are then further encapsulated in a stretchable outer shell, in which two films of thermoplastic polyurethane (TPU) are laminated and each side of the TPU is bonded with heat (150° C.). After that, the stretchable electronic strips are integrated into one of the textile channels through manual weaving. Every sensor is exposed through the opening and glued to the textile with a washable fabric glue. Four power and signal wires from the main hub are threaded to every end of these strips to connect the microprocessor to all available sensors.

In this prototype, one or more temperature sensors are embedded in a fabric and encapsulated by a thermally conductive epoxy and thermoplastic polyurethane.

In this prototype, in order to measure a user's cardiac waveform (or any aspect thereof, such as heart rate or heart rate variability), raw data is processed with a finite impulse response (FIR) low-pass filter with Fₛ of 1000 Hz, Fₚₐₛₛ of 60 Hz, Fₛₜₒₚ frequency of 180 Hz, Dₚₐₛₛ of 0.05, and Dₛₜₒₚ of 0.0001, where D is the deviation (ripple) vector. This pass filter eliminates low-frequency respiratory waveforms.

In this prototype, in order to measure a user's respiratory waveform, raw data is processed with a FIR low-pass filter with Fₛ of 1000 Hz, Fₚₐₛₛ of 1 Hz, Fₛₜₒₚ frequency of 2 Hz, Dₚₐₛₛ of 0.0005, and Dₛₜₒₚ of 0.000001. This filter eliminates high-frequency signals due to heart-beat events and helps obtain a DC component of the signals.

This prototype includes four temperature sensing modules, one inertial sensing module, and two interconnection modules. In an area of 25 cm×27.5 cm flexible board, a total of 66 temperature sensors and 20 interconnection strips may be located. The temperature sensor has an accuracy of 0.1° C. between 37 to 39° C., and a 0.0039° C. resolution, which is rounded up programmatically to 0.01° C. There may be up to 32 unique addresses for the temperature sensors, which may be set by connecting ground (GND), power supply (VDD), data line (SDA) or clock line (SCL) signal to the A0, A1, and A2 pins on the chip. Given that there are eight combinations possible in these three pins for each signal, there are four different hard-wired A0, A1, and A2 pins to voltage supply (VDD) or ground (GND) and data-line (SDA) or clock-line (SCL). Each temperature module may be connected by soldering the jumpers. The capacitor complement of the temperature sensor is used as a decoupling capacitor to stabilize the local VDD supply from high-frequency noise and voltage ripples. The mechano-acoustic sensor or inertial measurement unit (IMU) is capable of measuring 3-axis gyroscope and 3-axis accelerometer, with a programmable accelerometer range of ±2 g to 16 g, a highest precision of 0.00012 g or 0.0012 m/s2, and a maximum of two addresses in one I2C bus. Four pads at each side of all sensor modules connect to power and signal lines (VDD, SCL, SDA, GND).

The prototype described in the preceding 28 paragraphs is a non-limiting example of this invention. This invention may be implemented in many other ways.

### Customized Dimensions of Conformable Garment

As noted above, the dimensions of the garment may be customized (e.g., tailored) to help achieve a snug fit. To customize a garment for a particular user, measurements may be taken of the size of different parts of the particular user's body. For instance, the measurements may measure one or more of the following dimensions: front length of torso, back length of torso, neck circumference, armhole depth, armhole circumference, elbow circumference, wrist circumference, sleeve inseam, shoulder-to-bicep, bicep-to-elbow, elbow-to-wrist, shoulder-to-wrist, shoulder width back, center back, bust, upper waist circumference, waist, hip circumference, neck-to-bust, neck-to-upper-waist, neck-to-waist, neck-to-upper-hip, neck-to-hip, crotch-to-ankle, waist-to-ankle, knee circumference, and ankle circumference. These measured dimensions may be entered via one or more input/output devices (e.g., keyboard, mouse, or touch screen). Then, to achieve a snug, customized fit for a particular user, the garment may be fabricated in such a way that at least some of the dimensions of the garment are slightly smaller than the corresponding measured dimensions of the user's body. For instance, in some cases, at least some of the dimensions of the garment are between 2% and 30% smaller than the corresponding measured dimensions of the user's body.

### Software

In the Computer Program Listing above, three computer program files are listed. These three computer program files comprise software employed in a prototype of this invention.

In order to submit these three programs to the U.S. Patent and Trademark Office, the three program files were converted to ASCII .txt format. In each of these three programs, these changes may be reversed, so that the three programs may be run. Specifically, these changes may be reversed by deleting “.txt” from each filename extension and replacing it with “.py”.

This invention is not limited to the software set forth in these three computer program files. Other software may be employed. Depending on the particular implementation, the software used in this invention may vary.

### Computers

In illustrative implementations of this invention, one or more computers (e.g., servers, network hosts, client computers, integrated circuits, microcontrollers, controllers, microprocessors, processors, field-programmable-gate arrays, personal computers, digital computers, driver circuits, or analog computers) are programmed or specially adapted to perform one or more of the following tasks: (1) to control the operation of, or interface with, hardware components including any sensor, memory device or wireless module; (2) to process signals, (3) to analyze data that represents measurements taken by one or more sensors; (4) to perform any other calculation, computation, program, algorithm, or computer function described or implied herein; (5) to receive signals indicative of human input; (6) to output signals for controlling transducers for outputting information in human perceivable format; (7) to process data, to perform computations, and to execute any algorithm or software; and (8) to control the read or write of data to and from memory devices (tasks 1-8 of this sentence being referred to herein as the “Computer Tasks”). The one or more computers may each comprise: (a) a central processing unit, (b) an ALU (arithmetic logic unit), (c) a memory unit, and (d) a control unit that controls actions of other components of the computer in such a way that encoded steps of a program are executed in a sequence. In some cases, the one or more computers (e.g., 301, 1107, 1014) communicate with each other or with other devices: (a) wirelessly, (b) by wired connection, (c) by fiber-optic link, or (d) by a combination of wired, wireless or fiber optic links.

In exemplary implementations, one or more computers are programmed to perform any and all calculations, computations, programs, algorithms, computer functions and computer tasks described or implied herein. For example, in some cases: (a) a machine-accessible medium has instructions encoded thereon that specify steps in a software program; and (b) the computer accesses the instructions encoded on the machine-accessible medium, in order to determine steps to execute in the program. In exemplary implementations, the machine-accessible medium may comprise a tangible non-transitory medium. In some cases, the machine-accessible medium comprises (a) a memory unit or (b) an auxiliary memory storage device. For example, in some cases, a control unit in a computer fetches the instructions from memory.

In illustrative implementations, one or more computers execute programs according to instructions encoded in one or more tangible, non-transitory computer-readable media. For example, in some cases, these instructions comprise instructions for a computer to perform any calculation, computation, program, algorithm, or computer function described or implied herein. For instance, in some cases, instructions encoded in a tangible, non-transitory, computer-accessible medium comprise instructions for a computer to perform the Computer Tasks.

### Computer Readable Media

In some implementations, this invention comprises one or more computers that are programmed to perform one or more of the Computer Tasks.

In some implementations, this invention comprises one or more tangible, machine readable media, with instructions encoded thereon for one or more computers to perform one or more of the Computer Tasks. In some implementations, these one or more media are not transitory waves and are not transitory signals.

In some implementations, this invention comprises participating in a download of software, where the software comprises instructions for one or more computers to perform one or more of the Computer Tasks. For instance, the participating may comprise (a) a computer providing the software during the download, or (b) a computer receiving the software during the download.

### Network Communication

In illustrative implementations of this invention, one or more devices (e.g., 1014, 1020) are configured for wireless or wired communication with other devices in a network.

For example, in some cases, one or more of these devices include a wireless module for wireless communication with other devices in a network. Each wireless module (e.g., 1012) may include (a) one or more antennas, (b) one or more wireless transceivers, transmitters or receivers, and (c) signal processing circuitry. Each wireless module may receive and transmit data in accordance with one or more wireless standards.

In some cases, one or more of the following hardware components are used for network communication: a computer bus, a computer port, network connection, network interface device, host adapter, wireless module, wireless card, signal processor, modem, router, cables and wiring.

In some cases, one or more computers (e.g., 301, 1107, 1014) are programmed for communication over a network. For example, in some cases, one or more computers are programmed for network communication: (a) in accordance with the Internet Protocol Suite, or (b) in accordance with any other industry standard for communication, including any USB standard, ethernet standard (e.g., IEEE 802.3), token ring standard (e.g., IEEE 802.5), or wireless communication standard, including IEEE 802.11 (Wi-Fi®), IEEE 802.15 (Bluetooth®/Zigbee®), IEEE 802.16, IEEE 802.20, GSM (global system for mobile communications), UMTS (universal mobile telecommunication system), CDMA (code division multiple access, including IS-95, IS-2000, and WCDMA), LTE (long term evolution), or 5G (e.g., ITU IMT-2020).

### Definitions

The terms “a” and “an”, when modifying a noun, do not imply that only one of the noun exists. For example, a statement that “an apple is hanging from a branch”: (i) does not imply that only one apple is hanging from the branch; (ii) is true if one apple is hanging from the branch; and (iii) is true if multiple apples are hanging from the branch.

To compute “based on” specified data means to perform a computation that takes the specified data as an input.

The term “comprise” (and grammatical variations thereof) shall be construed as if followed by “without limitation”. If A comprises B, then A includes B and may include other things.

The term “computer” means a computational device that is configured to perform logical and arithmetic operations. Each of the following is a non-limiting example of a “computer”, as that term is used herein: (a) digital computer; (b) analog computer; (c) computer that performs both analog and digital computations; (d) microcontroller; (e) controller; (f) microprocessor; (g) processor; (h) field-programmable gate array; (i) tablet computer; (j) notebook computer; (k) laptop computer, (1) personal computer; (m) mainframe computer; (n) integrated circuit; (o) server computer; (p) client computer; and (q) quantum computer. However, a human is not a “computer”, as that term is used herein.

“Computer Tasks” is defined above.

“Defined Term” means a term or phrase that is set forth in quotation marks in this Definitions section.

For an event to occur “during” a time period, it is not necessary that the event occur throughout the entire time period. For example, an event that occurs during only a portion of a given time period occurs “during” the given time period.

The term “e.g.” means for example.

As used herein, to say that an object is “elongated” means that the object's length is at least ten times greater than its width.

The fact that an “example” or multiple examples of something are given does not imply that they are the only instances of that thing. An example (or a group of examples) is merely a non-exhaustive and non-limiting illustration.

Unless the context clearly indicates otherwise: (1) a phrase that includes “a first” thing and “a second” thing does not imply an order of the two things (or that there are only two of the things); and (2) such a phrase is simply a way of identifying the two things, so that they each may be referred to later with specificity (e.g., by referring to “the first” thing and “the second” thing later). For example, if a device has a first socket and a second socket, then, unless the context clearly indicates otherwise, the device may have two or more sockets, and the first socket may occur in any spatial order relative to the second socket. A phrase that includes a “third” thing, a “fourth” thing and so on shall be construed in like manner.

“For instance” means for example.

Each of the following is a non-limiting example of a “garment”, as that term used herein: bodysuit, suit, shirt, top, tank, pants, belt, sock, glove, undershirt, underpants, wrist-band, ankle-band, arm-band, leg-band, head-band, hat, cap, clothing, and textile wearable.

To say a “given” X is simply a way of identifying the X, such that the X may be referred to later with specificity. To say a “given” X does not create any implication regarding X. For example, to say a “given” X does not create any implication that X is a gift, assumption, or known fact.

“Herein” means in this document, including text, specification, claims, abstract, and drawings.

As used herein: (1) “implementation” means an implementation of this invention; (2) “embodiment” means an embodiment of this invention; (3) “case” means an implementation of this invention; and (4) “use scenario” means a use scenario of this invention.

The term “include” (and grammatical variations thereof) shall be construed as if followed by “without limitation”.

As used herein, an “inner surface” of a garment means a surface of the garment that is configured to face toward a user when the garment is worn by the user.

Unless the context clearly indicates otherwise, “or” means and/or. For example, A or B is true if A is true, or B is true, or both A and B are true. Also, for example, a calculation of A or B means a calculation of A, or a calculation of B, or a calculation of A and B.

Each of the following is a non-limiting example of a “physiological aspect” of a user: temperature in one or more regions of the user's body, heart rate, heart rate variability, cardiac waveform, pulse waveform, respiration rate, respiration waveform, electrodermal activity, brain electrical activity, neural electrical activity, muscle electrical activity, activity level, motion, and seismocardiographic motion.

A non-limiting example of a sensor “pressing against” skin occurs when an outermost layer (e.g., TPU or other encapsulant) of the sensor presses against the skin.

A human is not a “processor”, as that term is used herein.

A human is not a “sensor”, as that term is used herein.

As used herein, the term “set” does not include a group with no elements.

Unless the context clearly indicates otherwise, “some” means one or more.

As used herein, a “subset” of a set consists of less than all of the elements of the set.

The term “such as” means for example.

A non-limiting example of a sensor “touching” skin occurs when an outermost layer (e.g., TPU or other encapsulant) of the sensor touches the skin.

“TPU” means thermoplastic polyurethane.

To say that a machine-readable medium is “transitory” means that the medium is a transitory signal, such as an electromagnetic wave.

As used herein, to say that a sensor is “waterproof” means that the sensor includes one or more layers of material that are configured to prevent water from entering an interior region of the sensor, which interior region houses electronic components of the sensor.

Except to the extent that the context clearly requires otherwise, if steps in a method are described herein, then the method includes variations in which: (1) steps in the method occur in any order or sequence, including any order or sequence different than that described herein; (2) any step or steps in the method occur more than once; (3) any two steps occur the same number of times or a different number of times during the method; (4) one or more steps in the method are done in parallel or serially; (5) any step in the method is performed iteratively; (6) a given step in the method is applied to the same thing each time that the given step occurs or is applied to a different thing each time that the given step occurs; (7) one or more steps occur simultaneously; or (8) the method includes other steps, in addition to the steps described herein.

Headings are included herein merely to facilitate a reader's navigation of this document. A heading for a section does not affect the meaning or scope of that section.

This Definitions section shall, in all cases, control over and override any other definition of the Defined Terms. The Applicant or Applicants are acting as his, her, its or their own lexicographer with respect to the Defined Terms. For example, the definitions of Defined Terms set forth in this Definitions section override common usage and any external dictionary. If a given term is explicitly or implicitly defined in this document, then that definition shall be controlling, and shall override any definition of the given term arising from any source (e.g., a dictionary or common usage) that is external to this document. If this document provides clarification regarding the meaning of a particular term, then that clarification shall, to the extent applicable, override any definition of the given term arising from any source (e.g., a dictionary or common usage) that is external to this document. Unless the context clearly indicates otherwise, any definition or clarification herein of a term or phrase applies to any grammatical variation of the term or phrase, taking into account the difference in grammatical form. For example, the grammatical variations include noun, verb, participle, adjective, and possessive forms, and different declensions, and different tenses.

### Variations

This invention may be implemented in many different ways. Here are some non-limiting examples:

In some implementations, this invention is a method comprising: (a) exerting, with an inner surface of a garment, pressure against a user in such a way that at least a portion of the pressure is greater than 1 mmHg and less than 41 mmHg and is due to elastic stretching of the garment; (b) measuring, with a set of sensors housed in or on the garment, one or more physiological aspects of the user while the exerting of pressure occurs; and (c) sending, from each particular sensor in the set of sensors, electrical signals that travel to one or more processors via electrical wiring and that encode digital data which represents or is derived from measurements taken by the particular sensor; wherein (i) the one or more processors are separate from the set of sensors and are located in or on the garment, and (ii) each particular sensor in the set of sensors (A) includes an integrated chip, (B) is waterproof, (C) touches skin of the user during the measuring, and (D) either (I) is part of, or is attached to, or protrudes through the inner surface of the garment, or (II) is exposed to the skin of the user through one or more holes in the inner surface of the garment. In some cases, the method further comprises: (a) removing the one or more processors from the garment; (b) after the removing, washing the garment while the sensors remain in or on the garment; and (c) after the washing, returning the one or more processors to a region in or on the garment, which region had been occupied by the one or more processors before the washing. In some cases: (a) during the exerting of pressure, the garment is elastically strained; (b) the garment has a set of dimensions that occur when the garment is not undergoing elastic strain and is not being worn by the user; and (c) each dimension in the set of dimensions is smaller, by at least 5%, than a corresponding dimension of the user's body. In some cases: (a) the electrical wiring, via which signals are sent from the sensors to the one or more processors, includes a set of wires; (b) the garment includes a set of channels; and (c) at least a portion of each particular wire in the set of wires is located inside a hollow elongated region of a particular channel in the set of channels. In some cases, the method further comprises wirelessly transmitting signals which encode data that is outputted by the one or more processors and that is derived from measurements taken by the sensors. In some cases: (a) a power source comprises one or more batteries, is separate from the sensors, and is housed, together with the one or more processors, in a single housing that is located in or on the garment; and (b) the method further includes providing electrical power to the sensors from the one or more batteries. In some cases, the garment comprises a knitted textile. In some cases, the set of sensors includes different types of sensors, including at least one accelerometer and at least one temperature sensor. In some cases, the one or more physiological aspects that are measured include one or more of cardiac waveform, heart rate, heart rate variability or any other attribute of the cardiac waveform. In some cases, the one or more physiological aspects that are measured include respiration rate. Each of the cases described above in this paragraph is an example of the method described in the first sentence of this paragraph, and is also an example of an embodiment of this invention that may be combined with other embodiments of this invention.

In some implementations, this invention is a system comprising: (a) a garment; (b) a set of sensors housed in or on the garment; (c) electrical wiring housed in or on the garment; and (d) one or more processors; wherein (i) the one or more processors are separate from the set of sensors and are located in or on the garment, (ii) the set of sensors is configured to take measurements of one or more physiological aspects of the user, (iii) each particular sensor in the set of sensors (A) includes an integrated chip, (B) is waterproof, (c) is configured to send electrical signals that travel to the one or more processors via the electrical wiring and that encode digital data which represents or is derived from measurements taken by the particular sensor, and (D) either (I) is part of, or is attached to, or protrudes through the inner surface of the garment, or (II) is exposed through one or more holes in the inner surface of the garment, and (iv) the garment is configured to exert pressure, when worn by a user, against the user in such a way that (A) at least a portion of the pressure is greater than 1 mmHg and less than 41 mmHg and is due to elastic stretching of the garment, and (B) each sensor in the set of sensors touches skin of the user while the measurements are taken and the pressure is exerted. In some cases, the sensors are configured to remain, without being damaged, in or on the garment even while the garment is being washed. In some cases: (a) the garment is configured to be elastically stretched when worn by the user; (b) the garment is configured to have a set of dimensions when the garment is not undergoing elastic strain and is not being worn by the user; and (c) each dimension in the set of dimensions is smaller, by at least 5%, than a corresponding dimension of the user's body. In some cases: (a) the electrical wiring, via which signals are sent from the sensors to the one or more processors, includes a set of wires; (b) the garment includes a set of channels; and (c) at least a portion of each particular wire in the set of wires is located inside a hollow elongated region of a particular channel in the set of channels. In some cases: (a) the system further includes a transceiver; and (b) the system is configured to transmit, from the transceiver, wireless signals which encode data that is outputted by the one or more processors and that is derived from measurements taken by the sensors. In some cases: (a) the system includes a power source which comprises one or more batteries, is separate from the sensors, and is housed, together with the one or more processors, in a single housing that is located in or on the garment; and (b) the system is configured to provide electrical power to the sensors from the one or more batteries. In some cases, the garment comprises a knitted textile. In some cases, the set of sensors includes different types of sensors, including at least one accelerometer and at least one temperature sensor. In some cases, the one or more physiological aspects which the set of sensors is configured to measure include one or more of cardiac waveform, heart rate, heart rate variability, or any other attribute of the cardiac waveform. In some cases, the one or more physiological aspects which the set of sensors is configured to measure include respiration rate of the user. Each of the cases described above in this paragraph is an example of the system described in the first sentence of this paragraph, and is also an example of an embodiment of this invention that may be combined with other embodiments of this invention.

Each description herein (or in the Provisional) of any method, apparatus or system of this invention describes a non-limiting example of this invention. This invention is not limited to those examples, and may be implemented in other ways.

Each description herein (or in the Provisional) of any prototype of this invention describes a non-limiting example of this invention. This invention is not limited to those examples, and may be implemented in other ways.

Each description herein (or in the Provisional) of any implementation, embodiment or case of this invention (or any use scenario for this invention) describes a non-limiting example of this invention. This invention is not limited to those examples, and may be implemented in other ways.

Each Figure, diagram, schematic or drawing herein (or in the Provisional) that illustrates any feature of this invention shows a non-limiting example of this invention. This invention is not limited to those examples, and may be implemented in other ways.

The above description (including without limitation any attached drawings and figures) describes illustrative implementations of the invention. However, the invention may be implemented in other ways. The methods and apparatus which are described herein are merely illustrative applications of the principles of the invention. Other arrangements, methods, modifications, and substitutions by one of ordinary skill in the art are also within the scope of the present invention. Numerous modifications may be made by those skilled in the art without departing from the scope of the invention. Also, this invention includes without limitation each combination and permutation of one or more of the items (including any hardware, hardware components, methods, processes, steps, software, algorithms, features, and technology) that are described herein.

What is claimed:

1. A method comprising:
(a) exerting, with an inner surface of a garment, pressure against a user in such a way that at least a portion of the pressure is greater than 1 mmHg and less than 41 mmHg, and is due to elastic stretching of the garment;
(b) measuring, with a set of sensors housed in or on the garment, one or more physiological aspects of the user while the exerting of pressure occurs; and
(c) sending, from each particular sensor in the set of sensors, electrical signals that travel to one or more processors via electrical wiring and that encode digital data which represents or is derived from measurements taken by the particular sensor;
wherein
(i) the one or more processors are separate from the set of sensors and are located in or on the garment, and
(ii) each particular sensor in the set of sensors (A) includes an integrated chip, (B) is waterproof, (C) touches skin of the user during the measuring, and (D) either (I) is part of, or is attached to, or protrudes through the inner surface of the garment, or (II) is exposed to the skin of the user through one or more holes in the inner surface of the garment.

2. The method of claim 1, wherein the method further comprises:
(a) removing the one or more processors from the garment;
(b) after the removing, washing the garment while the sensors remain in or on the garment; and
(c) after the washing, returning the one or more processors to a region in or on the garment, which region had been occupied by the one or more processors before the washing.

3. The method of claim 1, wherein:
(a) during the exerting of pressure, the garment is elastically strained;
(b) the garment has a set of dimensions that occur when the garment is not undergoing elastic strain and is not being worn by the user; and
(c) each dimension in the set of dimensions is smaller, by at least 5%, than a corresponding dimension of the user's body.

4. The method of claim 1, wherein:
(a) the electrical wiring, via which signals are sent from the sensors to the one or more processors, includes a set of wires;
(b) the garment includes a set of channels; and
(c) at least a portion of each particular wire in the set of wires is located inside a hollow elongated region of a particular channel in the set of channels.

5. The method of claim 1, wherein the method further comprises wirelessly transmitting signals which encode data that is outputted by the one or more processors and that is derived from measurements taken by the sensors.

6. The method of claim 1, wherein:
(a) a power source comprises one or more batteries, is separate from the sensors, and is housed, together with the one or more processors, in a single housing that is located in or on the garment; and
(b) the method further includes providing electrical power to the sensors from the one or more batteries.

7. The method of claim 1, wherein the garment comprises a knitted textile.

8. The method of claim 1, wherein the set of sensors includes different types of sensors, including at least one accelerometer and at least one temperature sensor.

9. The method of claim 1, wherein the one or more physiological aspects that are measured include one or more of cardiac waveform, heart rate, heart rate variability or any other attribute of the cardiac waveform.

10. The method of claim 1, wherein the one or more physiological aspects that are measured include respiration rate.

11. A system comprising:
(a) a garment;
(b) a set of sensors housed in or on the garment;
(c) electrical wiring housed in or on the garment; and
(d) one or more processors;
wherein
(i) the one or more processors are separate from the set of sensors and are located in or on the garment,
(ii) the set of sensors is configured to take measurements of one or more physiological aspects of the user,
(iii) each particular sensor in the set of sensors
(A) includes an integrated chip,
(B) is waterproof,
(c) is configured to send electrical signals that travel to the one or more processors via the electrical wiring and that encode digital data which represents or is derived from measurements taken by the particular sensor, and
(D) either (I) is part of, or is attached to, or protrudes through the inner surface of the garment, or (II) is exposed through one or more holes in the inner surface of the garment, and
(iv) the garment is configured to exert pressure, when worn by a user, against the user in such a way that
(A) at least a portion of the pressure is greater than 1 mmHg and less than 41 mmHg and is due to elastic stretching of the garment, and
(B) each sensor in the set of sensors touches skin of the user while the measurements are taken and the pressure is exerted.

12. The system of claim 11, wherein the sensors are configured to remain, without being damaged, in or on the garment even while the garment is being washed.

13. The system of claim 11, wherein:
(a) the garment is configured to be elastically stretched when worn by the user;
(b) the garment is configured to have a set of dimensions when the garment is not undergoing elastic strain and is not being worn by the user; and
(c) each dimension in the set of dimensions is smaller, by at least 5%, than a corresponding dimension of the user's body.

14. The system of claim 11, wherein:
(a) the electrical wiring, via which signals are sent from the sensors to the one or more processors, includes a set of wires;
(b) the garment includes a set of channels; and
(c) at least a portion of each particular wire in the set of wires is located inside a hollow elongated region of a particular channel in the set of channels.

15. The system of claim 11, wherein:
(a) the system further includes a transceiver; and
(b) the system is configured to transmit, from the transceiver, wireless signals which encode data that is outputted by the one or more processors and that is derived from measurements taken by the sensors.

16. The system of claim 11, wherein:
(a) the system includes a power source which comprises one or more batteries, is separate from the sensors, and is housed, together with the one or more processors, in a single housing that is located in or on the garment; and
(b) the system is configured to provide electrical power to the sensors from the one or more batteries.

17. The system of claim 11, wherein the garment comprises a knitted textile.

18. The system of claim 11, wherein the set of sensors includes different types of sensors, including at least one accelerometer and at least one temperature sensor.

19. The system of claim 11, wherein the one or more physiological aspects which the set of sensors is configured to measure include one or more of cardiac waveform, heart rate, heart rate variability, or any other attribute of the cardiac waveform.

20. The system of claim 11, wherein the one or more physiological aspects which the set of sensors is configured to measure include respiration rate of the user.
